(19) United States
(12) Patent Application Publication
(10) Pub. No.: US 2001/0037101 A1
(43) Pub. Date: Nov. 1, 2001

(54) Tagged superabsorbent polymers in a multicomponent structure

(76) Inventors: David S. Allan, Midland, MI (US); Joseph L. Weir, Midland, MI (US); Richard M. Van Effen, Midland, MI (US); Sergio S. Cutie, Midland, MI (US)

Correspondence Address: THE DOW CHEMICAL COMPANY, INTELLECTUAL PROPERTY SECTION, P. O. BOX 1967, MIDLAND, MI, 48641-1967, US

(21) Appl. No.: 09/803,073

(22) Filed: Mar. 9, 2001

Related U.S. Application Data
(60) Provisional application No. 60/190,275, filed on Mar. 17, 2000.

(51) Int. Cl. A61F 13/15; A61F 13/20

(52) U.S. Cl. 604/368; 604/367; 604/385.01

(57) ABSTRACT

The present invention is an absorbent composite that contains at least two intermixed or segregated superabsorbent polymers, at least one of which is incorporated throughout with a latent indicator that becomes manifest by a developer that is peculiar to the indicator. The invention provides a means of monitoring the performance of accuracy of superabsorbent polymer placement in absorbent structures such as diapers, adult incontinence devices, and sanitary napkins.

### CROSS-REFERENCE TO RELATED APPLICATION

This application claims the benefit of United States Provisional Application No. 60/190275, filed on Mar. 17, 2000.

### BACKGROUND OF THE INVENTION

This invention relates to tagged superabsorbent polymers (SAPs) in multicomponent absorbent structures such as diapers, adult incontinence devices, and sanitary napkins.

Such absorbent structures may contain two or more kinds of SAPs, for example, a) a centrally located insult region (or contact region) containing an SAP of high permeability and slow absorption; and b) a retention region containing an SAP of low permeability and high retention, where the absorbed fluids are retained. The shape, size, and location of these regions are chosen to maximize the structure's effectiveness in carrying the fluids away from the point of insult and containing them in a separate region. See, for example, U.S. Pat. No. 5,728,082 (Gustafson et al.); EP 558,889 A1 (Roos et al.); U.S. Pat. No. 4,338,371 (Dawn et al.); EP 631,768 A1 (Pilschke); WO 98/22067 (Matthews et al.); WO 97/34558 (Schmidt); WO 98/06364 (Hsueh et al.); EP 640,330 A1 (Pilschke).

Manufacturing defects may result in improper placement of one or more of the SAPs, thereby reducing the effectiveness of the absorbent structure. However, since the SAPs are indistinguishable to the naked eye, such defects go unnoticed. It would therefore be an advance in the art to be able to quickly and easily monitor the performance of accuracy of SAP placement in absorbent structures.

### SUMMARY OF THE INVENTION

The present invention addresses a need by providing an absorbent composite comprising at least two superabsorbent polymers, either segregated or intermixed, wherein at least one of the superabsorbent polymers is incorporated throughout with a latent indicator that becomes manifest by a development method so that the incorporated superabsorbent can be distinguished from all other components upon development of the indicator.

In a second aspect, the present invention is an absorbent composite comprising at least two segregated or intermixed superabsorbent polymers, wherein each superabsorbent polymer, or one less than each superabsorbent polymer, is incorporated throughout with a latent indicator peculiar to that superabsorbent polymer so that each superabsorbent can be distinguished from each other upon development of the indicators.

In a third aspect, the present invention is a method of manifesting a superabsorbent polymer in an absorbent composite that contains at least two segregated or intermixed superabsorbent polymers, which method comprises the steps of: a) incorporating a latent indicator into a first superabsorbent polymer; b) preparing a patterned composite of the first superabsorbent polymer and a second superabsorbent polymer so that each superabsorbent polymer defines a particular region of the patterned composite; c) treating the patterned composite with a developer for the latent indicator so that the first superabsorbent polymer becomes manifest distinctly from the second superabsorbent polymers.

### BRIEF DESCRIPTION OF THE DRAWINGS

FIG. 1 is a top view illustration of a diaper containing two segregated superabsorbent polymers having distinct absorbing and retaining characteristics.

FIG. 2 is a side view of a diaper containing two superabsorbent polymers having distinct absorbing and retaining characteristics.

FIG. 3 is a top view illustration of a diaper containing a manufacturing flaw.

### DETAILED DESCRIPTION OF THE INVENTION

The absorbent composite of the present invention comprises a plurality of SAPs with distinct absorbing and retaining properties, wherein at least one of the SAPs contains a distinct latent indicator which becomes manifest through some development method. The SAPs may be segregated or intermixed.

As used herein, the term “latent indicator” refers to a material incorporated into the SAP that is invisible to the naked eye, but that responds to a chemical, electrical, thermal, or electromagnetic stimulus to become manifest. The term “Superabsorbent polymer region” is used herein to refer to a part of the absorbent composite that contains a particular SAP. Each region contains a different SAP.

SAPs are well known in the art and are described, for example, in U.S. Pat. No. 5,356,403, column 3, lines 39 et seq., which reference is incorporated herein by reference. Examples of commercially available SAPs include DRYTECH™ 2035 SAP (a trademark of The Dow Chemical Company), and IM-3900 SAP (available from Hoechst-Celanese Corporation).

Examples of suitable latent indicators include, but are not limited to (with the developing agent in parentheses), starch (iodine), iodine (starch), aluminum salts (aluminon), amine or ammonium salts (ninhydrin), magnesium or calcium salts (Eriochrome Black T), fluorescent agents (UV light), and pH sensitive indicators (acid or base). All that is required in the practice of the present invention is that each SAP be incorporated with a sufficiently distinct indicator (or no indicator at all) so as to allow the tester, upon development of the indicator, to discern the distinct superabsorbent regions in their entirety.

As suggested above, indicator need not be present in each distinct region; since the absence of a response is a response (in the same way that the absence of color is black), an untreated region can be readily distinguished from a treated one. Thus, in a diaper having two distinct SAP regions, it is possible, and preferable, to incorporate throughout one of the regions a latent indicator, while incorporating no indicator into the other. (Similarly, where three distinct regions exist, it is preferable to incorporate distinct latent indicators into two of the regions, while leaving the third region without indicator.)

The indicator can be incorporated into the SAP by a variety of methods including dry-blending, slurry addition, spraying, or by addition to the SAP monomer precursor followed by polymerization of the monomer to the SAP by any suitable method such as those well known in the art. The concentration of the indicator in the SAP is that amount sufficient to manifest the incorporated region upon development. For sensitive techniques (e.g., iodine indicator developed by starch development), part per million levels are sufficient; in less sensitive techniques (e.g., ainine or ammonium salt indicator developed by ninhydrin) low percent levels, preferably from about 0.5, more preferably from about 1 percent, to about 10, more preferably to about 5 percent are used. One of ordinary skill in the art would know what quantities of indicator to use without undue experimentation.

Once the indicator is incorporated into the SAP of interest, the polymer composite is prepared. In this manner, the incorporated region or regions can be readily distinguished. The present invention can be more readily understood by reference to the illustrative embodiments.

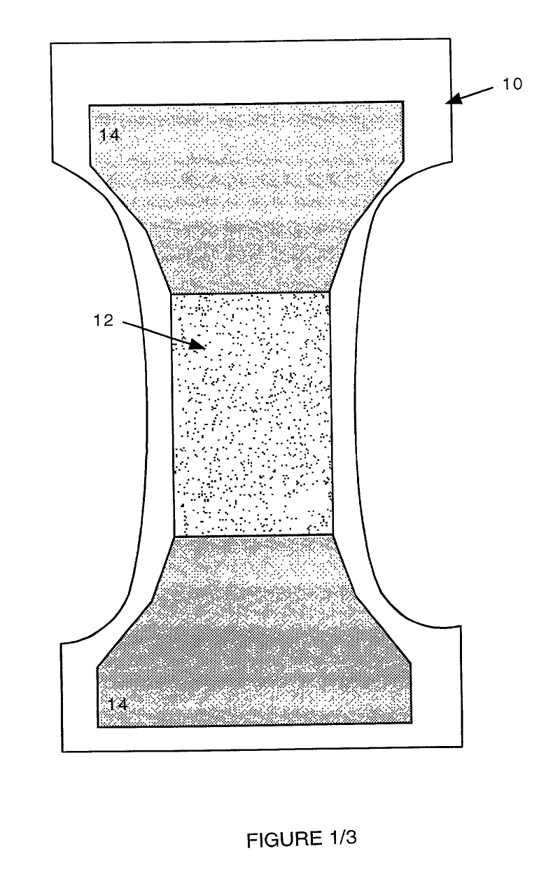

In FIG. 1, which is a top view illustration of a preferred embodiment of the present invention, the diaper (10) contains two distinct superabsorbing regions, the insult region (12), which contains an SAP of high permeability and slow absorption and the retention region (14), which contains an SAP of low permeability and high retention. The insult region (12) is also incorporated throughout with a latent indicator (12a), while the retention region (14) is not incorporated with any indicator. (Note, the SAP that is treated with latent indicator is shown as dots for the purposes of illustration. In practice, the indicator cannot be seen except after development.) Thus, the undeveloped diaper appears monochromatic (usually white), while the developed diaper manifests the size, shape, and location of the SAP of interest.

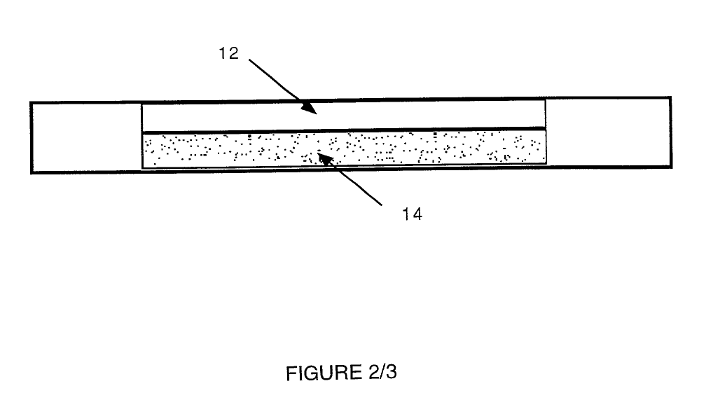

In FIG. 2, which is a side view illustration of another embodiment of the present invention, the insult region (12) and retention region (14) appear as stacked layers. In this embodiment, the retention region contains an SAP of high permeability and slow absorption (12), but no incorporated indicator. Only the retention region (14) contains the latent indicator (14a).

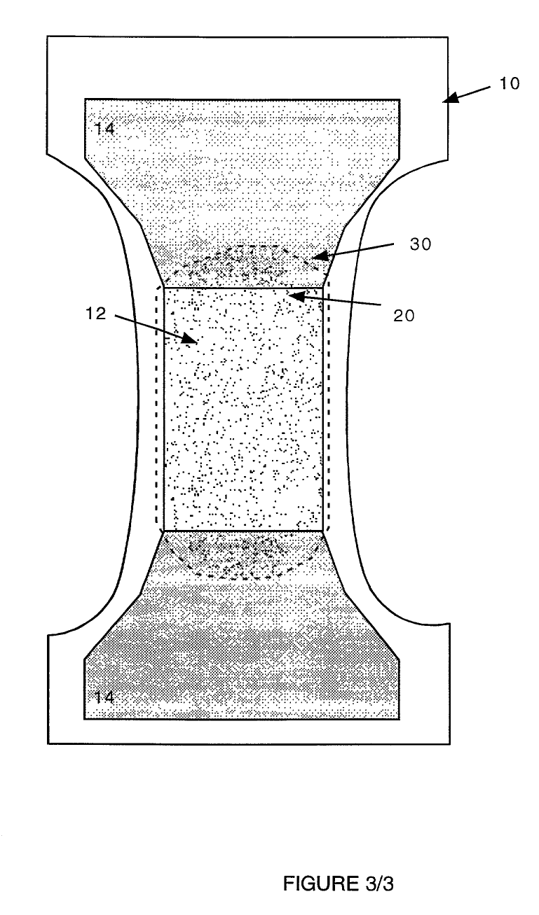

FIG. 3 is illustrative of the phenomenon of bleeding. In this illustration, the insult region (12) contains a latent indicator (12a) but the retention region (14) does not. The solid lines (20) separating the insult region (12) from the retention region (14) indicate the intended configuration of the diaper whereas the dotted lines (30) indicate the actual configuration that is the result of a manufacturing defect. The development of the latent indicator (12b) reveals this defect and allows for its detection before the diaper is packaged and sent to its final destination.

The present invention provides an efficient way to detect manufacturing flaws in the production of multicomponent SAP composites such as diapers, adult incontinent products, feminine hygiene products, wound dressings, and the like.

The following examples are for illustrative purposes only and are not intended to limit the scope of this invention.

### EXAMPLE 1

### Starch-iodine Dry Blend Process

A. Preparation of the starch-treated SAP

Conventional SAP (DRYTECH 2035 SAP, 1200 g) is loaded into a laboratory Forberg mixer. Commercial corn starch powder (60 g, 5 parts per hundred parts SAP (pph)) is added and the powders are dry blended for one minute. The blender is opened and the walls are scraped free of powder. Aluminum sulfate solution (alum, 48% by weight, 48 g of solution) is added through a two-fluid spray nozzle over a period of one minute while blending the solids. The blender is stopped after alum addition, the walls are scraped, and the product is blended an additional five minutes. This starch-treated SAP has an absorbancy under 0.9 psi load (0.9 psi AUL) of greater than 15 g/g.

B. Preparation of the patterned composite containing a treated SAP and an untreated SAP

A patterned composite is made with the starch-treated SAP and an untreated SAP having a 0.9 psi AUL of less than 15 g/g. The composite is made by air-laying 8.5 g of cellulose fluff and 11.6 g (total) of SAP in a laboratory absorbent composite pad former. The pad dimensions are 14-in long×4-in (36-cm long×10-cm). About half of the cellulose fluff (˜4 g) is laid down on a tissue substrate. Low-0.9 psi AUL superabsorbent is laid in two one-inch stripes (2.5-cm) along the long edge of the pad, leaving the center two inches (5 cm) of the pad free of SAP. The high-0.9 psi AUL, starch-labeled SAP is laid down in the central two-inch×14-inch (5-cm×36-cm) stripe. The remainder of the fluff is laid on top of the SAP layer. The pad is densified to a thickness of about ⅛ inch (0.3 cm) in a laboratory press between platens heated to about 100° C. The composite is sprayed with a 0.005N solution of iodine in 0.9% aqueous sodium chloride. The starch-treated SAP is easily distinguished from the untreated SAP by its blue color. The contrast between the two SAPs intensifies as the composite dries out.

### EXAMPLE 2

### Starch-Iodine Slurry Process

The patterned composite is prepared as in Example 1 except that the starch-treated SAP is made by a slurry process. In this process, a slurry of cornstarch is prepared by mixing alum (48%, 20 g) with water (60 g), then adding starch (50 g, 5 pph) with good stirring in a Waring blender. VORANOL 2070 polyether polyol (0.5 g) is then added to this slurry for dust control. SAP is loaded into the laboratory Forberg mixer. The mixer is turned on and the slurry is sprayed onto the SAP through a two-fluid nozzle. The mixer is opened and the walls scraped down. The product is then blended for another five minutes. The final product is screened through a 20 mesh screen, with about 0.2-0.3% retained on the screen as oversize.

### EXAMPLE 3

### Iodine-Starch Inverse Process

The patterned composite is prepared as described in Example 1 except that the latent indicator is iodine, and the developer is starch. In this process, a solution consisting of water (5.38 g), VORANOL 2070 poly(propylene oxide) (2.69 g), and potassium iodide (1.92) is prepared. SAP (1200 g) is placed into the laboratory Forberg blender. A portion of the solution (0.6 g) is sprayed onto the SAP to achieve a potassium iodide loading of 100 ppm based on SAP. The blender is stopped and a 100-g aliquot of treated SAP is removed.

The color in the SAP is developed by treating the patterned composite with stabilized starch indicator solution, followed by 30% hydrogen peroxide solution. (Treatment first with hydrogen peroxide, followed by treatment with starch, is equally effective).

### EXAMPLE 4

### Collagen-Ninhydrin

The patterned composite is prepared as described in Example 1 except that the latent indicator is collagen. Collagen hydrolysate (Kraft Foods, Woburn AGH-1 hydrolysate Type A, 40 g) is dissolved in water (80 g) by first dispersing the collagen in methanol, then adding water and boiling off the methanol with stirring. The Forberg blender is loaded with 1200 g of the SAP, and 12 g of collagen solution is sprayed onto the SAP through a two-fluid nozzle. Very little agglomeration of SAP is observed. A developing solution is prepared by dissolving ninhydrin (1 g) in ethanol (7.5 mL), then adding ethyl acetate (15 mL) and acetic acid (0.3 mL). Finally, this solution is added to n-heptane (50 mL). The composite is sprayed with ninhydrin developing solution, then heated in a steam bath to develop the blue color.

### EXAMPLE 5

### Aspartic Acid in Monomer Mix-Ninhydrin

The patterned composite is prepared as described in Example 1 except that the latent indicator is aspartic acid. In this instance 1-wt % aspartic acid is added to the acrylic acid monomer is a standard polymerization reaction as follows. A solution containing acrylic acid (340.5 g), water (349.7 g), VERSENEX 80 chelating agent (a trademark of The Dow Chemical Company 0.43 g), ethoxylated trimethylolpropane triacrylate (Sartomer, 1.7 g), sodium chlorate (1.4 g), and L-aspartic acid (4.14 g) is neutralized partially by slow addition of a solution of sodium carbonate (162.9 g) in water (407.3 g). The monomer mix so prepared is charged into a water jacketed two-liter glass reactor equipped with high torque agitator, nitrogen sparge, and vacuum/nitrogen attachment. The monomer mix is sparged with nitrogen for 60 minutes, then heated to 30° C. Hydrogen peroxide (0.11 g of 30% solution) and sodium persulfate (5.45 g of 10% solution) are added and the mixture stirred for two minutes. Sodium erythorbate (0.51 g of 10% solution) is added after the stirring time. The temperature of the water jacket is adjusted to maintain pseudo-adiabatic conditions. When the polymerized gel reached 85° C., vacuum is applied to the headspace to prevent the gel temperature from exceeding 90° C. The gel is held in the reactor for three hours at 65° C. under mild agitation, during which time the gel is ground into a crumb approximately one centimeter in diameter. The gel is dried for 30 min at 165° C. in a forced-air dryer, then ground in a two-roll mill and sieved to retain that portion which passes through a 20-mesh screen and is retained on a 120-mesh screen. Color is developed as described in Example 4.

### EXAMPLE 6

### Aluminum Sulfate-Aluminon

The patterned composite is prepared as described in Example 1 except that the latent indicator is aluminum sulfate. The SAP is treated with aluminum sulfate (48% solution, 6% treatment by spraying on the surface of the SAP in a laboratory blender). Color is developed by treating the composite with a solution of aluminon (aurine tricarboxylic acid, ammonium salt, 0.05%), methanol (50%), and saline (5% concentration, 49.05%).

### EXAMPLE 7

### Ammonium Sulfate in Monomer Mix-Ninhydrin

Ammonium sulfate is added to the monomer mix (1%, based on the weight of acrylic acid) instead of aspartic acid in a standard polymerization reaction as described in Example 5. Color is developed as described in Example 4.

### EXAMPLE 8

### Magnesium Carbonate-Eriochrome Black T

Magnesium carbonate (5 pph) is dry-blended with the SAP. A solution of Eriochrome Black T (200 mg), triethanolamine (15 mL), and absolute ethanol (5 mL) is prepared as a developing reagent. Treatment of the SAP with developing solution yielded a faint pink color.

### EXAMPLE 9

### Optical Brighteners-UV light

Liquid optical brightener (Tinopal DCL Liq. New 450#, Prod. #0925091UX, Ciba) is sprayed at a loading of 2%. Exposure of the composite to long wavelength UV light exposes the treated region.

What is claimed is:

1. An absorbent composite comprising at least two superabsorbent polymers, either segregated or intermixed, wherein at least one of the superabsorbent polymers is incorporated throughout with a latent indicator that becomes manifest by a development method so that the incorporated superabsorbent can be distinguished from all other components upon development of the indicator.

2. The absorbent composite of claim 1 wherein the superabsorbent polymers are segregated.

3. The absorbent composite of claim 1 wherein the superabsorbent polymers are intermixed.

4. The absorbent composite polymer of claim 2 wherein one of the superabsorbent polymers is incorporated throughout with the indicator selected from the group consisting of starch, iodine, an aluminum salt, an amine or ammonium salt, a magnesium or calcium salt, a fluorescent agent, and a pH sensitive indicator.

5. The absorbent composite polymer of claim 4 wherein the absorbent composite polymer comprises two superabsorbent polymers.

6. The absorbent composite polymer of claim 5 wherein the indicator is selected from the group consisting of starch and an amine or ammonium salt.

7. An absorbent composite comprising at least two segregated or intermixed superabsorbent polymer regions, wherein each superabsorbent polymer, or one less than each superabsorbent polymer, is incorporated throughout with a latent indicator peculiar to that superabsorbent polymer so that each superabsorbent polymer can be distinguished from each other upon development of the indicators.

8. A method of manifesting a superabsorbent polymer region in an absorbent composite that contains at least two segregated or intermixed superabsorbent polymers, which method comprises the steps of a) incorporating a latent indicator into a first superabsorbent polymer; b) preparing a patterned composite of the first superabsorbent polymer and a second superabsorbent polymer so that each superabsorbent polymer defines a particular region of the patterned composite; c) treating the patterned composite with a developer for the latent indicator so that the first superabsorbent polymer becomes manifest distinctly from the second superabsorbent polymers.

9. The method of claim 8 wherein the composite contains two segregated superabsorbent polymers.

10. The method of claim 8 wherein the latent indicator is selected from the groups consisting of starch, iodine, an aluminum salt, an amine or ammonium salt, a magnesium or calcium salt, a fluorescent agent, and a pH sensitive indicator.

11. The method of claim 9 wherein the latent indicator is starch and the developer is iodine.

12. The method of claim 9 wherein the latent indicator is iodine and the developer is starch.

13. The method of claim 9 wherein the latent indicator is an aluminum salt and the developer is aluminon.

14. The method of claim 9 wherein the latent indicator is an amine or ammonium salt and the developer is ninhydrin.

15. The method of claim 9 wherein the latent indicator is a magnesium or a calcium salt and the developer is Eriochrome Black T.

16. The method of claim 9 wherein the latent indicator is a fluorescent agent and the developer is UV light.

17. The method of claim 9 wherein the latent indicator is a pH sensitive indicator and the developer is an acid or a base.

18. The method of claim 8 wherein the latent indicator is incorporated in the first superabsorbent polymer by adding the indicator to a monomer precursor for the first superabsorbent polymer, then polymerizing the monomer.

19. The method of claim 8 wherein the latent indicator is incorporated by dry-blending.

20. The method of claim 8 wherein the latent indicator is incorporated by spraying a solution or slurry of the indicator onto the first superabsorbent polymer.

21. The method of claim 8 wherein the latent indicator is a thermally sensitive indicator and the developer is heat.
